(19) United States
(12) Patent Application Publication
(10) Pub. No.: US 2003/0074083 A1
(43) Pub. Date: Apr. 17, 2003

(54) Femoral neck prosthesis

(76) Inventors: Brian N. LeGros, Chatham (CA); Markus Frohlich, Balterswil (CH); Urs P. Wyss, Raterschen (CH); Heidy-Lynn Ploeg, Ottawa (CA)

Correspondence Address: CHRISTIE, PARKER & HALE, LLP, P.O. BOX 7068, PASADENA, CA, 91109-7068, US

(21) Appl. No.: 10/235,232

(22) Filed: Sep. 5, 2002

(30) Foreign Application Priority Data
Sep. 10, 2001 (EP) 01 810 866.2

(51) Int. Cl. A61F 2/36

(52) U.S. Cl. 623/23.35; 623/22.44; 623/22.46

(57) ABSTRACT

The invention relates to a femoral neck prosthesis having a spherically shaped head (1), by which a centre (2) of a sphere is defined, and having a stem (3) which extends from the level of the centre (2) of the sphere along a centre line (4) over a length (L) up to a distal end (5). Since the stem has a modulus of elasticity E of between 10 GPa and 60 GPa, since the length L lies between 50 mm and 150 mm and since the equivalent stiffness S=E L formed from the product of the modulus of elasticity E and the length L lies between 0.5·10⁹ and 9·10⁹ [Newton/metres], the stem behaves similarly to the bone material itself and the shear strains between the stem and the bone material are kept low.

The invention relates to a femoral neck prosthesis having a spherically shaped head, by which a centre of a sphere is defined, and having a stem which extends from the level of the centre of the sphere along a centre line over a length L up to a distal end.

A femur endoprosthesis having a short implantation length and a bent stem end which abruptly tapers caudally is shown in the patent specification U.S. Pat. No. 6,120,544. The stem end and the upper region of the prosthesis, which can be inserted without using cement, are covered with a three-dimensional spatial net structure in order to allow better ingrowth of bone material. The usual metals for implantations are provided as materials, as for a pressure disc prosthesis. In comparison with the bone material into which it is embedded, such a construction has a substantially greater stiffness at the interface to the bone material, which necessarily results in unwanted high shear stresses.

It is the object of the present invention to form a protective transfer of the forces from the prosthesis to the bone material surrounding it. This object is satisfied in that the stem has a modulus of elasticity E of between 10 GPa and 60 GPa; in that the length L is between 50 mm and 150 mm; and in that the equivalent stiffness S=E L formed from the product of the modulus of elasticity E and the length L is between 0.5 10⁹ and 9 10⁹ [Newton/metres].

Large regions of the upper femoral bone are exposed to a similar strain as under natural conditions by the adaptation of the modulus of elasticity E of the prosthesis to that of the bone material surrounding it while taking into account a length L of the prosthesis which lies between 150 mm and 50 mm. The stem behaves similarly to the bone material itself. At the same time, the loss of bone material is kept small.

Dependent claims 2 to 15 represent advantageous further developments of the invention.

It is thus advantageous to make the stem with a curvature of the centre line in the frontal plane which becomes tighter in the direction distal to the femoral axis in order to open into the femoral axis or to merge into the femoral axis with a kink so that a good approximation to the natural shape of the femoral bone takes place. This is further improved if the stem has elliptical cross-sections which reduce from proximal to distal and which are rotated relative to one another about an angle of rotation of ε≦8° along the centre line over an angle of curvature α of, for example, 45°.

Furthermore, the outer surface of the stem can have a first contact surface at the points undergoing large strain, that is beneath the head medially at the stem, which is led close up to the cortex and extends parallel to this, as well as a second contact surface with respect to the cortex of the femur in the femoral medullary space laterally at the stem in order to transfer a tilt moment of the stem to the bone. These contact surfaces, which are provided directly for the contact to the cortex, have an area from 150 to 450 mm² in order to keep the specific pressure low. A further possibility for the taking up of loading forces consists of a collar directly adjoining the head and sitting on the resected femoral neck, with the collar not necessarily being perpendicular to the centre line of the stem. Rather, the plane in which the collar lies is optimised with respect to the forces taken up due to its position such that a specific position is associated with this plane such that this plane forms an angle with its normal of β=87°±3° in the projection onto the frontal plane with respect to a horizontal X axis extending towards medial, an angle of γ=15°±3° with the vertical Z axis in the projection onto the sagittal plane and an angle of δ=13°±3° with the horizontal X axis towards medial in the projection onto the transversal plane.

In practice only non-metallic materials, that is plastics, or plastics reinforced with fibre material or fillers, can be considered as the stem material with a modulus of elasticity of between 10 GPa and 60 GPa. The modulus of elasticity of 3.5 GPa of pure PEEK can brought to a value of 150 GPa mixing in such fibre materials and fillers. The following table lists by way of example possible plastics, their tensile strength and their modulus of elasticity with respect to tensile strain.

| | Tensile strength | Modulus of |
|---|---|---|
| Material | (MPa) | elasticity (GPa) |
| PEEK (polyetheretherketone) | 93.8 | 3.5 |
| PEI (poletherimide) | 105 | 3.0 |
| PET (polyethyene terephtha- | 159 | 8.96 |
| late | | |
| PPS (polyphenylene sulphide) | 138 | 11.7 |
| PAEK (polyaryletherketone | 121 | 9.0 |

These can be reinforced by one of the following materials in the form of fibres or powder in order to bring the modulus of elasticity up to a desired value:

Glass, glass fibres, carbon, carbon or graphite fibres respectively, organic compounds, boron, silicon carbide or ceramics.

Fibre materials can, however, also be embedded in the stem body as a fabric or a braid.

Since the head of the prosthesis must satisfy other criteria as a bearing, it can be a good idea to make the head from another material, for example as a hollow metal sphere or as a ceramic sphere, with there being the possibility of either parts of the head penetrating the stem or parts of the stem penetrating the head in order to make a good connection between the two components.

The invention will be described in the following with reference to embodiments. There are shown:

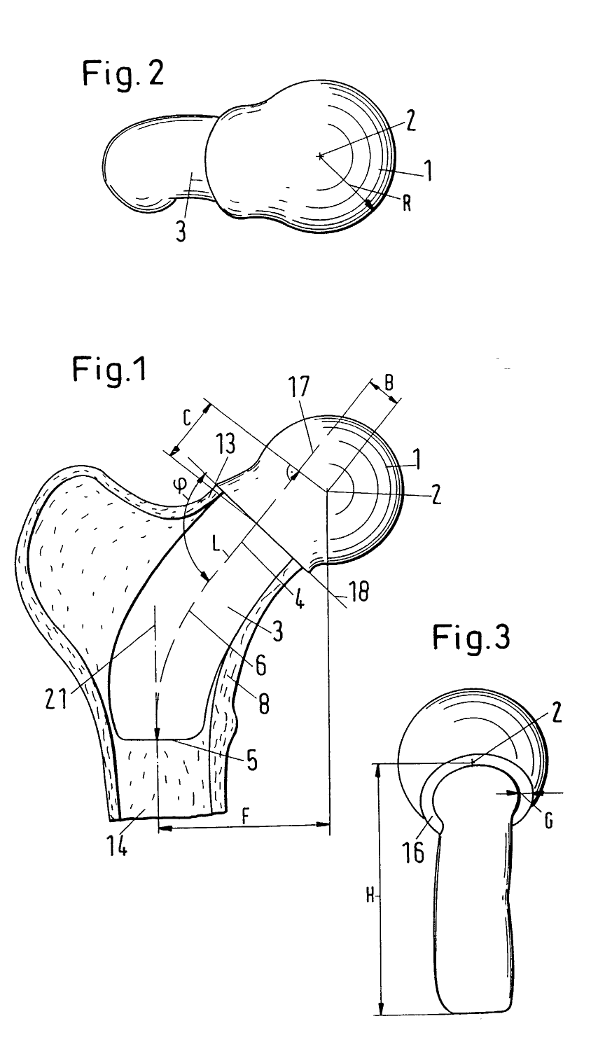

FIG. 1 schematically, a view from anterior of a prosthesis in accordance with the invention in a femoral bone imaged in the frontal plane;

FIG. 2 schematically, the prosthesis of FIG. 1 viewed from proximal;

FIG. 3 schematically, the prosthesis of FIG. 1 viewed from lateral;

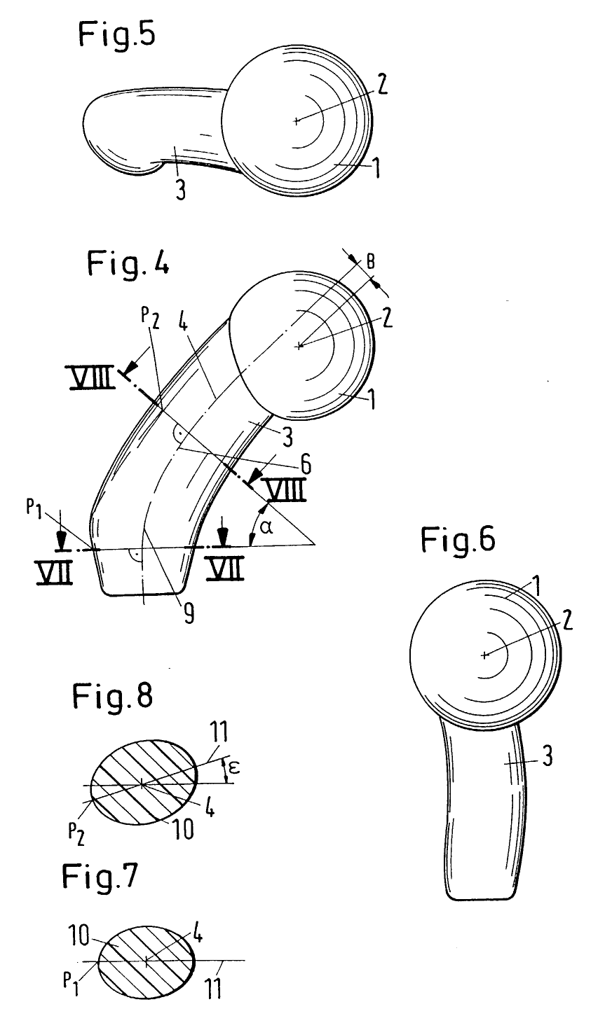

FIG. 4 schematically, a further embodiment of a femoral neck prosthesis without a collar viewed from anterior;

FIG. 5 schematically, the prosthesis of FIG. 4 viewed from proximal;

FIG. 6 schematically, the prosthesis of FIG. 4 viewed from medial;

FIG. 7 schematically, a section VII-VII in FIG. 4;

FIG. 8 schematically, a section VIII-VIII in FIG. 4;

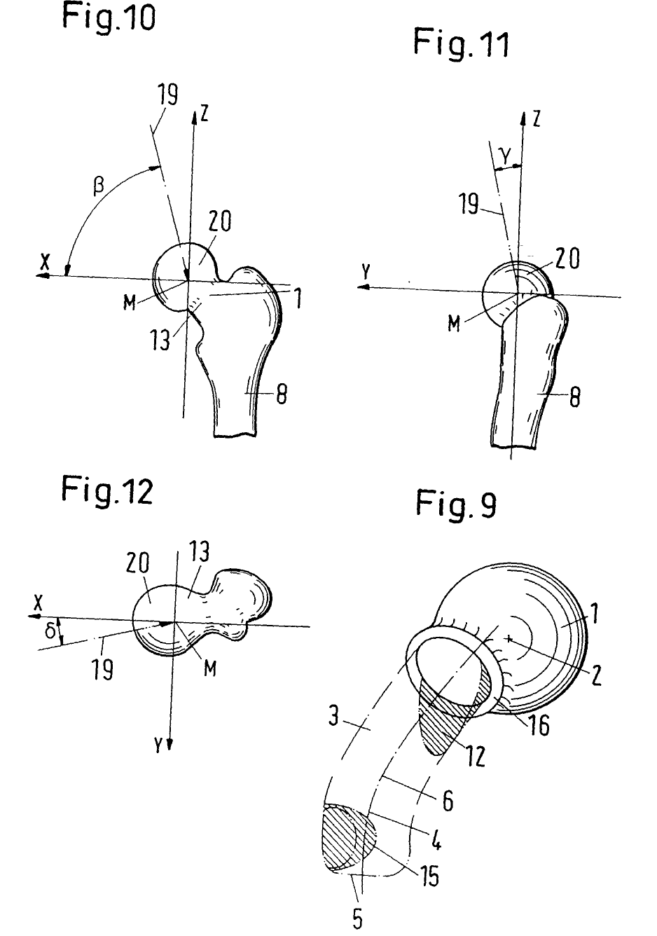

FIG. 9 schematically, a view from anterior and lateral of a prosthesis analogue to FIG. 1, in which the first and second contact surfaces are drawn in;

FIG. 10 schematically, in the frontal plane from anterior, the preferred angular position for the plane of the collar of a left femoral neck prosthesis with a collar support, represented by a normal which goes through the centre of a natural femoral head;

FIG. 11 schematically, the normal of FIG. 10 in its projection onto a sagittal plane viewed from lateral;

FIG. 12 schematically, the normal of FIG. 10 in its projection onto a transversal plane viewed from proximal;

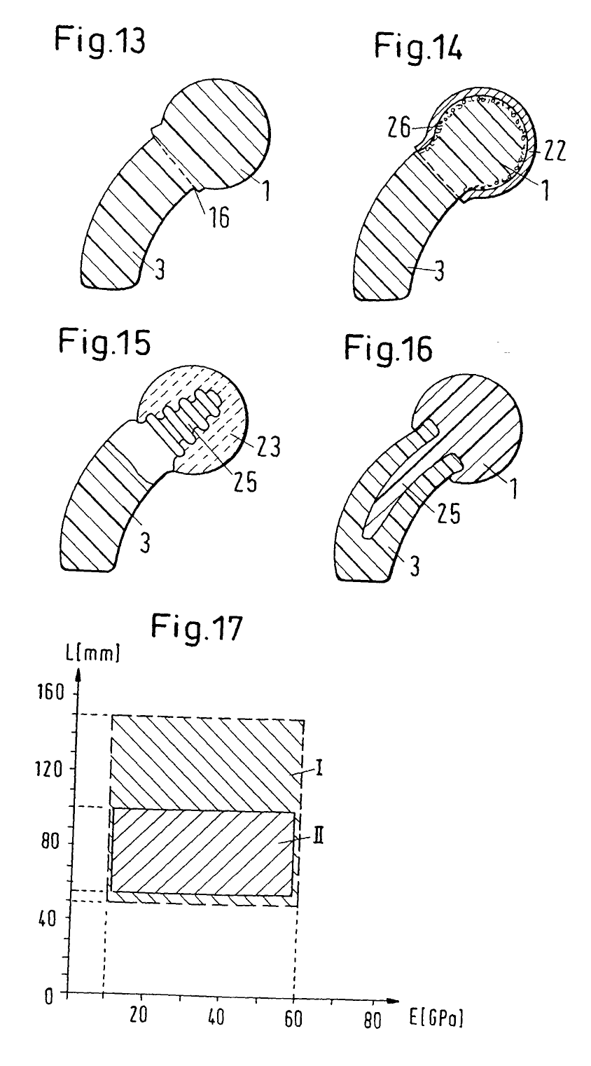

FIG. 13 schematically, a longitudinal section in the frontal plane through a femoral head prosthesis in which the head and the stem have a same polymer;

FIG. 14 schematically, a longitudinal section through a prosthesis analogue to FIG. 13, in which the bearing surface of the head is formed by a metallic hollow sphere;

FIG. 15 schematically, a longitudinal section through a prosthesis analogue to FIG. 13, in which the stem projects into the spherically shaped head for anchoring;

FIG. 16 schematically, a longitudinal section through a prosthesis analogue to FIG. 13, in which the spherical head projects into the stem for anchoring to an extension; and

FIG. 17 schematically, a graph in which the modulus of elasticity, the prosthesis length L and the preferred regions of the invention are entered.

The same reference symbols are used for the same functions in the Figures.

In an example of use shown in FIGS. 1, 2 and 3, the geometry of a femoral neck prosthesis in accordance with the invention is shown whose distal end 5 ends at the level of the trochanter minor. The centre 2 of the femoral head 1 has an overhang F of 45 mm with respect to the longitudinal axis 21, which can amount to between 30 and 55 mm. The centre line 4 of the stem 3 merges via a curvature 6 from the femoral axis 21 into the femoral neck 13. The stem merges into a collar 16 which is supported via a plane 18 formed by the resection surface. The centre 2 of the sphere is displaced towards medial by a spacing B of 7 mm, which can be between 3 and 12 mm, with respect to an extension 17 of the centre line and laid towards proximal by a spacing C of 18 mm, which can be between 12 to 26 mm. The radius R of the sphere, which can be 13 to 30 mm, was fixed at 24 mm for this example. The stem end 5 has a perpendicular spacing H of 65 mm, which can be between 50 and 85 mm, with respect to the sphere centre 2. The contact surface of the collar 16, like the plane 18, is inclined by an angle φ of 100° with respect to the centre line 4 in the frontal plane. The collar 16 has a width G of 3.5 mm, which can be from zero to 6 mm. The length L of the stem extends along the centre line 4 and its extension 17 from the stem end 5 up to the height of the centre 2 of the sphere. The modulus of elasticity for this stem (see graph FIG. 17) can lie between 10 and 60 GPa (giga Pascal). The stem itself has an elliptical shape for the cross-sections which reduce in the distal direction and can have a rotation through an angle ε≦8° along its centre line 4, as is described in the following embodiment.

In a further embodiment in FIGS. 4, 5, 6, 7 and 8, a femoral neck prosthesis without a collar is shown having a centre 2 of the sphere which is likewise displaced towards medial by a distance B from the extended centre line 4. The centre line 4 extends towards the distal end in a gentle curvature 6 and makes a slight kink 9 at the transition into the direction of the femoral axis. In accordance with FIG. 7, the distal end has an elliptical cross-section whose large axis 11 is in the frontal plane and passes laterally through a point P₁. When one moves upwards in FIG. 4 from this cross-section along the curved centre line 4 by an angle α of 45°, an elliptical, somewhat larger cross-section (FIG. 8) is again present there whose large axis 11 is rotated about an angle ε≦8° such that a point P₂ corresponding to point P₁ comes to rest more towards anterior on the large axis 11. The curvature of the stem exists not only in the frontal plane and an ante-version results for the spherical head 1. The modulus of elasticity for the stem likewise lies between 10 and 60 GPa.

In FIG. 9, the spatial position of a first contact surface 12 and of a second contact surface 15, which are provided particularly close to the cortex, or adjacent to these, is indicated by phantom lines. The bone bed is prepared particularly carefully in these regions. The first contact surface 12 lies towards medial and adjoins the collar 16 proximally and approximately forms a triangular area when unwound, i.e. in developed form. The second contact surface 15 is arranged towards lateral at the distal stem end and has the form of a patch which surrounds the stem by not quite 180°. In the regions of these contact surfaces, it is possible to keep the modulus of elasticity somewhat higher than at the remaining stem surface. The contact surfaces 12, 15 can have an extent from 150 to 450 mm².

The material of the stem can be plastics such as are listed in the table above and whose modulus of elasticity is increased by powder or fibres of a different material. For such a solution, it is interesting that powdered plastics, which for example have a higher melting point, but approximately the same modulus of elasticity as the base material, can also increase the overall modulus of elasticity when they arc distributed in a dispersed manner. In FIG. 13, a femoral neck prosthesis is shown whose stem 3 and head 1 are made in one piece of the same material, for example of plastic with powder fillers. The actual bearing surface at the head of the sphere is covered with a protective layer so that no fillers project directly into the surface. Manufacturing can be by thermoplastic injection and reworking at the head of the sphere.

In the femoral neck prosthesis in FIG. 14, a prefabricated spherical cap 22, for example made of metal on the inside, is covered with an open-pored layer 26 and subsequently injected with a thermoplastic charged with fillers, which simultaneously also forms the stem.

In the femoral neck prosthesis in FIG. 15, the spherical head 1 consists of a ceramic sphere 23 with a cavity into which a projecting spigot 25 of the stem 3 projects. The contact surface between the spigot 25 and the ceramic sphere 23 is provided with projections and recesses for securing.

In the femoral neck prosthesis in FIG. 16, the head 1 likewise consists of a different material having a higher modulus of elasticity than that shown by the stem 3. A spigot 25 projects from the head 1 into the stem 3 along the centre line into the stem in order to effect a better force transfer between the head and the stem.

In FIG. 17, two regions for the invention are entered. A first region for femoral neck prostheses having a length L between 50 and 150 mm and having a modulus of elasticity between 10 and 60 GPa results in equivalent stiffnesses of S=E·L which lie between 0.5·10⁹ and 9·10⁹ [New-ton/metres]. A second preferred region for femoral neck prostheses which are short in the majority and which have a length L between 55 and 100 mm and a modulus of elasticity between 10 and 60 GPa results in equivalent stiffnesses between 0.55 10⁹ and 6 10⁹ [Newton/metres].

In FIGS. 10, 11 and 12, the normal 19 with respect to the resection plane, which is drawn through the centre M of a natural femoral head 20 to be replaced, shows how a resection cut can be made in order to accept a collar considered favourable for the force transfer. The normal 19 stands in the frontal plane (FIG. 10) at an angle β of 87°±3° with respect to the X axis, in the sagittal plane (FIG. 11) at an angle γ of 15°±3° with respect to the Z axis and in the transversal plane (FIG. 12) at an angle δ of 13°±3° with respect to the X axis. To locate the correct position for the resection cut, the position of the normal 19 can first be fixed with a pre-bore through the femoral head 20 and subsequently the level for a resection cut perpendicular to the marked normal 19 can be determined.

The axes X, Y, Z are defined in their exact position with respect to the femoral bone in accordance with G. Bergmann [Habilitationsschrift Freie Universität Berlin 1994; Dr. Köster, Berlin 1997 (post-doctoral dissertation, Berlin Free University, 1994; Dr. Köster, Berlin 1997)]: “The forwardly bent centre line of the femur exits the bone at a point at distal between the condyles. In the proximal femur, the femur centre line and the femoral neck axis intersect approximately at a further point. A straight line is placed through these two points which is termed the longitudinal axis of the femur or the femoral axis. It is simultaneously the +z axis of the coordinate system oriented to proximal. A parallel is defined to the knee joint axis by the exit point in the condyle region. In this way, the knee axis is defined by the centres of the approximately semi-circular dorsal condyle portions in the sagittal plane.

The displaced knee joint axis and the femoral axis define the frontal plane of the femur. In this plane, the +x axis of the coordinate system directed to medial is perpendicular to +z and is not exactly parallel to the knee axis. The +y axis is fixed perpendicular to +x and +z. Its positive direction extends to the front for the left and right hip joint. Thus, an orthogonal right handed coordinate system is present at the left hand side and, in contrast, a left handed one is present at the right hand side. Thus, (x y), the transversal plane of the bone is fixed, and (y z) defines the sagittal plane.”

1. A femoral neck prosthesis having a spherically shaped head (1), by which a centre (2) of a sphere is defined, and having a stem (3) which extends from the level of the centre (2) of the sphere along a centre line (4) over a length (L) up to a distal end (5), characterised in that the stem has a modulus of elasticity E of between 10 GPa and 60 GPa; in that the length L lies between 50 mm and 150 mm; and in that the equivalent stiffness S=E·L formed from the product of the modulus of elasticity E and the length L lies between 0.5 10⁹ and 9 10⁹ [Newton/metres].

2. A femoral neck prosthesis in accordance with claim 1, characterised in that the centre line (4) of the stem (3) has a curvature (6).

3. A femoral neck prosthesis in accordance with claim 1, characterised in that the stem (3) has a downward kink (9) at its end (5) in the direction of the longitudinal axis (7) of a femur (8).

4. A femoral neck prosthesis in accordance with claim 1, characterised in that the stem (3) has elliptical cross-sections (10) perpendicular to the centre line (4) up to its distal end (5) whose areaurface reduces as they approach distal.

5. A femoral neck prosthesis in accordance with claim 4, characterised in that the large axes (11) of the elliptical cross-sections (10) perform a rotation about a rotational angle of ε≦8° with the shift from proximal toward distal.

6. A femoral neck prosthesis in accordance with claim 1, characterised in that a first contact surface (12) is provided for the cortex of a resected femoral neck beneath the head (1) medially at the stem (3) and is formed approximately as a triangle when unwound; and in that a second contact surface (15) is provided for the cortex of the femoral bone (8) for the region of the femoral medullary space (14) laterally at the stem (3) in order to transfer a tilt movement of the stem (3) to the bone (13, 8).

7. A femoral head prosthesis in accordance with claim 6, characterised in that the first and second contact surfaces (12, 15) each correspond to an area from 150 mm² to 450 mm².

8. A femoral head prosthesis in accordance with claim 1, characterised in that the stem (3) has a collar (16) towards the head (1).

9. A femoral head prosthesis in accordance with claim 8, characterised in that the collar (16) lies in a plane (18) with which a certain position is associated such that the plane (18) forms an angle with its normal (19) of α=87° in the projection onto the frontal plane with respect to a horizontal X axis extending towards medial, an angle of β=15° with the vertical Z axis in the projection onto the sagittal plane and an angle of γ=13° with the horizontal axis extending towards medial in the projection onto the transversal plane, with the angular figures being able to have a tolerance of plus/minus 3°.

10. A femoral head prosthesis in accordance with claim 1, characterised in that the head (1) and the stem (3) consist of different materials.

11. A femoral head prosthesis in accordance with claim 10, characterised in that the head (1) and the stem (3) each consist of a non-metallic material.

12. A femoral head prosthesis in accordance with claim 11, characterised in that the head (1) consists of a ceramic material.

13. A femoral head prosthesis in accordance with claim 10, characterised in that the head (1) is made as a hollow metal sphere into which a stem (3) made of plastic is moulded.

14. A femoral head prosthesis in accordance with claim 10, characterised in that the head (1) has an extension (17), to which a stem (3) is moulded.

15. A femoral head prosthesis in accordance with claim 1, characterised in that the stem (3) is reinforced by fibre material or fillers in order to set the modulus of elasticity to a desired value.
